(19) United States
(12) Patent Application Publication
(10) Pub. No.: US 2017/0303858 A1
(43) Pub. Date: Oct. 26, 2017

(54) HANDHELD BIOMETRIC SENSOR FOR MOBILE DEVICES

(71) Applicant: SENSIFREE LTD., Petach Tikva (IL)

(72) Inventors: Ilan Saul BARAK, Kfar Saba (IL); Eran AGMON, Sunnyvale, CA (US)

(73) Assignee: SENSIFREE LTD., Petach Tikva (IL)

(21) Appl. No.: 15/513,120

(22) PCT Filed: Jan. 5, 2016
(86) PCT No.: PCT/IB2016/050038

§ 371 Date: Mar. 21, 2017

Related U.S. Application Data
(60) Provisional application No. 62/101,934, filed on Jan. 9, 2015.

(51) Int. Cl. A61B 5/00; A61B 5/021

(52) U.S. Cl. CPC A61B 5/6898; A61B 5/02125; A61B 2562/0228

(57) ABSTRACT

A system and method comprises structure for receiving in a handheld device biometric or environmental data from the vicinity of the handheld device. The system may include a handheld mobile device such as a cellular telephone, or that device and a separate mechanical housing configured be held in a hand of a user and to clamp to or contain the handheld device. The system may include a specialized radar unit and be designed to analyze radar signals to provide a signal indicative of time varying arterial blood pressure. The mechanical housing may include a receiver designed to receive a biometric or environmental signal and to wirelessly transmit a corresponding signal to the handheld device when clamped to the handheld device.

This application claims the benefit of and priority to U.S. provisional application 62/101,934, filed Jan. 9, 2015, referenced as SF4 and SENS0013-1, which is incorporated herein by reference. All publications and documents, including but not limited to, patents, patent applications, articles, webpages, and books referenced herein below are herein incorporated by reference herein, in their entirety.

### SUMMARY OF THE INVENTION

The invention provides systems and methods for determining arterial blood pressure of a user as a function of time, in which the system a handheld structure comprising at least one of: (1) a handheld device configured to be held in one hand of a person comprising a cellular telephone transceiver and human interface; and (2) a handheld mechanical housing configured to be held in one hand of the person, and designed to removably mechanically attach or clamp onto a handheld device; a frequency controlled oscillator to generate RF signals; at least one antenna configured to transmit the RF signals generated by the frequency controlled oscillator and configured to receive RF signals which are reflections of the transmitted RF signals; a mixer configured to mix at least one of the transmitted RF signals with at least one of the reflected RF signals to generate a modified signal; an analog-to-digital converter configured to convert the modified signal into digital data thereby providing a digital representation of said modified signal; at least one processor for processing digital data; wherein said handheld structure contains said frequency controlled oscillator; said at least one antenna; and said mixer; said analog-to-digital converter; and said at least one processor; wherein said system is configured to: use said digital representation of said modified signal and a model relating said modified signal to arterial blood pressure, to generate an arterial blood pressure signal indicating arterial blood pressure of the user as a function of time. The methods of using the system comprise performing the configured use with a model to determine arterial blood pressure of the user as a function of time.

The invention also comprises systems and methods for transmitting data to a handheld device comprising a cellular telephone, in which the system comprises: a handheld structure comprising a handheld mechanical housing configured to be held in one hand of the person, and designed to removably mechanically attach or clamp onto a handheld device comprising a cellular telephone transceiver and a human interface; said handheld mechanical housing comprises a low power transceiver for wirelessly communicating with said handheld device; said handheld mechanical housing comprises a second transceiver for configured to receive an electromagnetic signal in a first frequency band. The methods of using the invention comprise attaching the handheld mechanical housing to the handheld device using the system for receiving the electromagnetic signal in the handheld mechanical system and wirelessly transmitting a signal related to that received signal to the handheld device.

### ASPECTS OF THE INVENTION

Aspects of the invention comprise: wherein said handheld device contains said frequency controlled oscillator; said at least one antenna; and said mixer; said analog-to-digital converter; and said at least one processor; wherein said handheld mechanical housing and wherein said handheld mechanical housing contains said frequency controlled oscillator; said at least one antenna; and said mixer, and a transceiver designed to wirelessly communicate with handheld device; wherein both said handheld device and said handheld mechanical housing and wherein said handheld mechanical housing contains said frequency controlled oscillator; said at least one antenna; and said mixer, and a low power transceiver designed to wirelessly communicate with handheld device; wherein said low power transceiver is designed to transmit in one of the following frequency bands: 6.765 to 6.795 MHZ; 13.553 to 13.567 MHZ; 26.957 to 27.283 MHZ; 40.660 to 40.700 MHZ; 433.050 to 434.790 MHZ; 902.000 to 928.000 MHZ; 2.400 to 2.500 GHz; 5.725 to 5.875 GHz; 24.000 to 24.250 GHz; 61.000 to 61.500 GHz; 122.000 to 123.000 GHz; and 244.000 to 246.000 GHz; wherein said low power transceiver is configured to transmit either said modified signal or said digital representation of said modified signal to said handheld device when said handheld device is removably clamped onto or contained in said handheld mechanical housing; wherein said oscillator is configured to generate signals having a bandwidth of at least 3 GHz in a frequency range between 3.0 GHz to 10.7 GHz; wherein said mixer comprises a single Schottky diode; wherein said mixer is configured to couple the RF signals to at least one antenna of said at least one antenna; wherein said at least one antenna comprises a first antenna and a second antenna, said first antenna is located in said handheld structure relative to an artery in a left hand of the user so that it senses arterial diameter when said system is held in the left hand of said user, and said second antenna is located in said handheld structure relative to an artery in a right hand of the user so that it senses arterial diameter when said system is held in the right hand of said user; wherein said system is configured to transmit said second signal corresponding to the time varying arterial blood pressure over a cellular network; wherein said electromagnetic signal is a biometric signal containing biometric information about a person holding said handheld mechanical housing; wherein said electromagnetic signal contains environmental information about an environment in the vicinity of said handheld mechanical housing; and wherein said handheld mechanical housing is removably mechanically attached or clamp onto said handheld device; and a heart rate determination algorithm designed to determine heart rate of a user, wherein said heart rate determination algorithm is designed to determine heart rate of a user from said digital representation of said modified signal and at least one model function for heart rate, wherein said algorithm is implemented in at least one of software and hardware.

### SUMMARY OF SOME OF THE EMBODIMENTS

Embodiments of apparatuses, methods and systems integrating contactless biometric sensors comprising antennae in a mobile device platform, such as a smartphone, are disclosed herein. In some embodiments, the disclosed methods, apparatuses and systems discuss embodiments of an ultra-wideband miniaturized thin antenna implementation of biometric sensors designed to detect biometric data from arteries close to a skin surface. Examples of these apparatuses comprise a mobile platform, that is, a handheld structure sized and designed to be held by one hand of a person, such as a smartphone or a handheld mechanical housing designed to clip on or attach to a smart phone. The apparatus may detect signals indicative of arterial diameters in the palm. In some instances, the antenna may be constructed using rigid flex PCB technology to achieve conformability, broadband capability, low cost and/or unidirectional radiation characteristics.

### BRIEF DESCRIPTION OF THE DRAWINGS

The drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1 shows a mobile device comprising some embodiments of the apparatus disclosed herein held in the palm of a hand and properly positioned above the superficial palmar arch.

FIG. 2 shows a cross section of the example mobile device of FIG. 1 depicting biosensing antennae contained in the mobile device.

FIG. 3 shows an example embodiment of an ultra-wideband microwave signal antenna used for sensing, for example, arteries.

FIG. 4 shows a cross section of a mobile device comprising some embodiments of the apparatus disclosed herein held in the palm of a hand with the biosensing antennae housed in a separate casing, that is a handheld mechanical housing.

FIG. 5 shows an electrical block diagram of an embodiment of mechanical housing 409 for use with mobile device 102.

FIG. 6 shows an electrical block diagram of an embodiment of mobile device 102 for use without mechanical housing 409.

FIG. 7 shows with a series of theoretical blood pressure versus time curves.

### DETAILED DESCRIPTION OF EMBODIMENTS

It has been shown previously that radar technology may be used to invasively estimate the arterial diameter of humans or animals. For example, PCT patent application publication WO/2013/118121, titled “A Microwave Contactless Heart Rate Sensor,” filed on Feb. 7, 2013, the entire contents of which are incorporated by reference herein, discloses a system that senses changes in arterial diameter via radiating radio frequency (RF) fields into tissue that includes an artery. Embodiments of the present disclosure may include features disclosed herein in combination with features disclosed in WO/2013/118121.

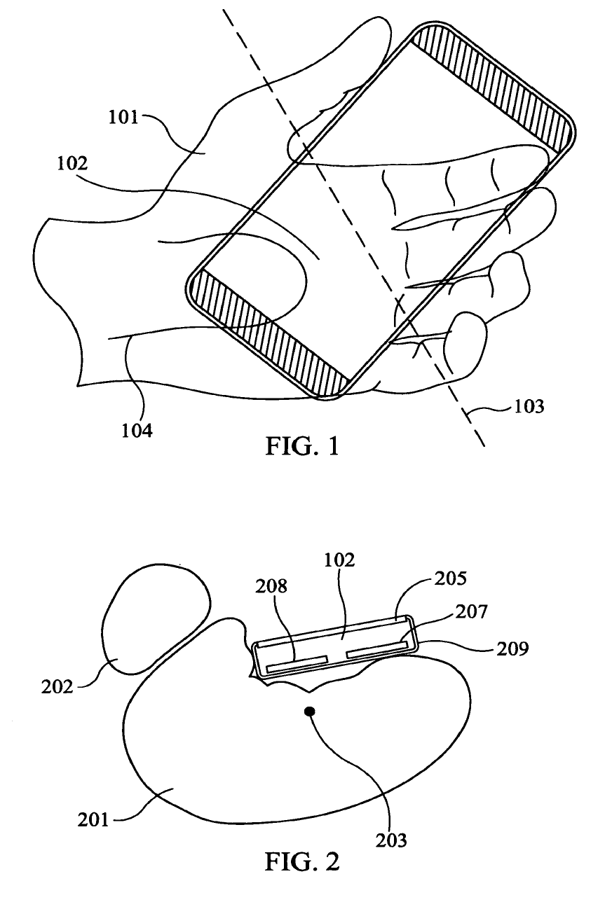

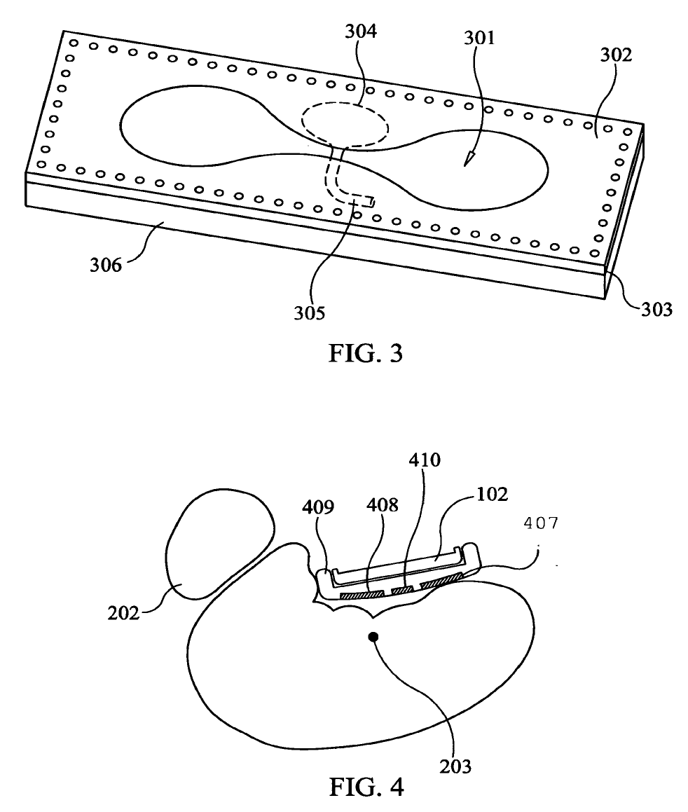

With reference to FIG. 1, in some embodiments, a good quality heart-rate and blood pressure detection may be obtained when the artery to be sensed is close to the skin surface. For example, the superficial palmar arch is close to the skin surface on the palm of a human body. The superficial palmar arch can be sensed by a device of the embodiments disclosed herein when the device is in the palm of a person's hand. FIG. 1 depicts a mobile device 102 held in the palm of a hand 101 wherein the superficial palmar arch 104 runs below the mobile device 102. The mobile device radiates a signal. Antenna in the mobile device, such as the antenna shown in FIG. 3, receive a reflection signal which are reflections by structure in the hand, including the arteries, of the radiated signal. The reflected signal contains signal indicative of the variation in size of the artery as a function of time. However, the instantaneous blood pressure in an artery is proportional to the instantaneous diameter of the artery. Therefore, the arterial signal also corresponds to and can be used to help determine arterial pressure as a function of time. As such, the blood pressure in the artery can be estimated. U.S. Patent Application Ser. No. 62/024,403, titled “Systems and Methods for Contactless Arterial Pressure Estimator”, filed Jul. 14, 2014, incorporated by reference herein in its entirety, describes methods for estimating blood pressure in an artery by measuring the instantaneous arterial diameter.

For example, in some embodiments, the radiated signal may be transmitted at a repetition rate sufficient to capture the changes in the artery diameter throughout the heart pulse cycle.

The heart rate of a person is on the order of 30 to 200 beats per minute. The radiated signal has a repetition rate. This repetition rate is sufficiently above the heart rate to provide reflection signal data points allowing for curve fitting the data to form a useful approximation for periodic variations in artery diameter. This data point acquisition rate may be for example, greater than 5 Hz, preferably greater than 15 Hz, and preferably between 50 to 1000 Hz. A preferred embodiment has a data acquisition rate of 200 to 280 Hz. From this data the heart rate can easily be extracted.

We can model the arterial pressure, p(t) as a function of the reflected signal strength, referred to herein below as the Pressure Wave, and as Sartery(t), as follows:

Sartery(t)=α*p(t)+K

Where Sartery(t) is this signal strength, p(t) is the artery pressure, a is a calibration constant, and K is a constant associated with the signal reflected from artery in the unrealistic condition where the arterial pressure is zero, and t is time. This signal may also be highly dependent on the antenna spacing from a skin surface, i.e., the palm, from each portion of the artery contributing to signal received by the antennas, and upon the relative orientation of each portion of the artery contributing signal received by the antennas relative to the orientation of the antennas. In some embodiments, the dimension as well as the antenna orientation in relation to the limb may vary during calibration or measurement, introducing significant measurement errors.

In some embodiments, such experimental/measurement errors may be compensated by estimating the distance of the antenna or antennas from the skin using the amplitude and phase of the signal reflected of other tissue layers (e.g., the skin layer, which produces the strongest echo) and using this estimate to modify the Sartery value, so the result is compensated against relative antennae-limb movements. For example, a look up table of the ratio of polynomials of the reflection amplitude and phase from various tissue layers may be prepared, and by interpolation, the shifts in position and/or orientation of the antennae may be determined and the resulting measurement errors compensated. In some embodiments, the errors may also be compensated by relying on the fact that the Pressure Wave peak-peak magnitude is invariant to the limb position, and as such, if the detected Sartery varies between the measurements, this may be attributed to the sensor-limb relative position change caused by the limb movement. In such embodiments, the variations may be calibrated out.

However, in any of the calibration cases, the calibration constant “α” is still to be determined. In some embodiments, one may determine the calibration constant α by measuring Sartery at a multiplicity of different arterial pressures where the difference in pressure is known (e.g., the constant K drops out in the difference since it is a constant) and relates only to the hydrostatic pressure difference. For example, the pressure difference may be created by the antenna in the subject hand being raised. This corresponds to a change height, ΔH, of the hand holding the antenna, relative to the height of the heart of the user resulting. Such change in height ΔH, leads to a shift in the arterial pressure wave change ΔP given by the expression ΔP=ρ*g*ΔH where ρ is the blood specific gravity, g is the gravitational acceleration constant. When the subject lifts his/her hand from the vertical downwards position to the vertical upwards position, ΔH can either be specified by the user, or it can be assumed to have some value. The assumed value for ΔH may depend upon known quantities related to the user, if known, such as the arm length, height, and gender of the user. In humans, there exists a practically fixed proportion between the body height and limb lengths. In some embodiments, the height difference can be estimated by including an accelerometer or gyro that are part of the sensing device, and integrating the vertical acceleration to estimate the vertical shift. Further, the height can be approximated by using an optical camera embedded in the device that, by using the height, length, etc., data on the subject, can then estimate the vertical shift. Values for p and g are known. Therefore, ΔP can be determined once ΔH is determined. The constant α=ΔS/ΔP, where ΔS is the time averaged difference in Pressure Wave at lower and higher positions of the wrist. Therefore, the constant “α” may also be determined once ΔH is known.

In some embodiments, the value a can be assumed to be a function of the Pressure Wave (e.g., as opposed to a constant), and in such embodiments, calibration can be done at many elevation positions of the arm holding the mobile device to approximate the nonlinear characteristic of Sartery as a function of the arterial pressure. From the several values of Sartery, in some embodiments, a distinct injective mapping of signal Sartery with the blood pressure may be obtained. In some embodiments, the calibration may utilize other acceleration sources in addition to gravity. For example, if the limb is accelerated by the subject by deliberate movement, this acceleration can be measured by an accelerometer, and the resulting change in Sartery can be correlated to the accelerometer measurement to extract the calibration constant a.

In some embodiments, the constant K may be determined as follows. For example, in some instances, the distinct peak representing Systolic pressure Pₛ and distinct valley for the Diastolic pressures P_{d} cannot be estimated without determining K, although the precise measurement PP, which is defined as the difference between the Systolic and Diastolic pressures, can be from a compensated Sartery signal approximating a representation of the Pressure Wave described in calibrated pressure units. In some embodiments, the shape of the pressure tail approximates, and may be represented by, an exponential curve. This pressure tail is a portion of the periodic arterial pressure cycle in which the pressure in the artery is decreasing towards its diastolic (minimum) value. This exponential form is believed to be a consequence of the pressure rate of change being linearly related to the pressure difference between the artery pressure and the vein pressure (and the vein pressure may be assumed to be a constant in some instances).

The pressure tail of Sartery(t) may be modeled as an exponentially decaying function. That model functional form may be curve fit to the actual data for the pressure tail. That is a best fit of the data Sartery(t)=α*p(t)+K to the model of the tail portion equal to “αP(t)+K”. For example, we obtain a best fit by minimizing norm 2 error. The determination of constants in the model for P(t) allow us to determine values for Pₛ and P_{d} due to the relationship of Pₛ and P_{d} and the constants in the model for P(t).

One model functional form for the pressure tail portion of p(t), of Sartery(t) is:

P(t)=P₀+P₁*e^{-P2(t-t0)}

Where “t” is time; P₁; P2; are known due to the exponential matching and t0 is a constants. We assume P0 is the venous (vein) pressure. The arterial pressure tail at infinite time is the vein pressure, P₀. Therefore:

Pₛ=P₀−P₁ (the systolic pressure)

P_{d}=P₀+P₁*e^{P2(Period-t0)}

Where “Period” is the periodicity of heart beats.

In some embodiments, the matching function may be an exponential decaying oscillating function representing the non-uniform frequency characteristic of the artery tree. In such embodiments, matching at additional points may enable the extraction of the function parameters, and allow for estimating the absolute values of Ps and Pd. In some embodiments, the calibrated pressure wave may be translated to the pressure as would be measured in the brachial artery and/or the central aorta blood pressure using a model of the artery tree. In some embodiments, this model may be a spectral model, and the mathematically equivalent time domain model may also be used.

With reference to FIG. 2, in some embodiments, a cross section of the example mobile device of FIG. 1 depicting biosensing antennae contained in the mobile device is shown. The cross section is along the cut line 103 shown in FIG. 1. In FIG. 2, the left hand palm cross section 201 is positioned palm-up and is viewed towards the fingers, with the thumb 202 shown for completeness. The mobile device 102 cross section reveals the screen 205 and the mobile device body shell 209. Two antennas 207 and 208 are positioned so that one antenna (e.g., 208) can sense the artery 203 diameter when places in one hand (right or left), then the other antenna (e.g., 207) performs similar function when held in the opposite hand. In some instances, the RADAR circuit may be connected to one or both antennae, so that the user may readily switch the mobile device between the left and the right hands without informing the mobile device in which hand it is held.

In some embodiments, the transmit and receive antenna design follows some of the embodiments described in U.S. Patent Application Ser. No. 62/083,981, titled “Systems, Apparatuses and Methods for Biometric Sensing Using Conformal Flexible Antenna”, filed Nov. 25, 2014, incorporated by reference herein in its entirety. However, as the installation inside a rigid mobile device may not require flexibility, its design can be simplified as shown in FIG. 3. The dielectric substrate comprises two materials, bottom dielectric sheet 306 and top dielectric sheet 303. The feed line 305 may be a conductive layer, feeding the shaped slot via capacitive coupling accomplished by elliptical disk 304. In some instances, the antenna may be fed by a microstrip line 305 on the top dielectric 303 (e.g., the top dielectric 303 may be a flexible PCB material and the microstrip line 305 is printed on the inner surface of the flexible PCB material), the line terminated by a capacitive disk 304 printed on the same surface. The disk may have one of a variety of shapes, an example of which is an elliptic shape, which may allow for a shorter connecting line on the far side of the slot 301. In addition, a detector diode, which may be part of the device, may be located on the antenna PCB half, minimizing the electrical distance between it and the antenna.

In some embodiments, the arterial sensor may comprise an antenna, an oscillator, a mixer, a converter, a processor, and/or and a battery for powering said components of the sensor. In some instances, the oscillator may be frequency controlled and may be utilized to generate the RF signal that is radiated by the antenna onto the hand tissue, such as the palm, which may result in signals being reflected from the tissue. In some instances, the mixer may mix the signal generated by said oscillator and the reflected signals received by the antenna reflected from said tissue.

In some implementations, the processor may generate a signal corresponding to the magnitude of the reflected signal from a body tissue. In some instances, the signal generated by the processor may correspond to the magnitude of the mixed signal from the mixer. For example, an analog-to-digital converter may sample the mixed and/or received signal, and the processor may split the sampled data into a plurality of bins, wherein each bin corresponds to a target located at a unique depth in the tissue and represents a magnitude of a reflected signal on said target, an example of such target tissue being an artery in the hand. As such, the signal generated by the processor from the mixed and/or the received signals may be used to identify tissues on the path of the signals. In some embodiments, for good differentiation between the different tissue parts, a wide bandwidth is used. For example, in some embodiments, at least 3 GHz is used to separate an artery reflection from a reflection from skin. In some embodiments, a higher bandwidth, e.g., 5 or 7.5 GHz, is advantageous.

For example, upon generating the signal, the processor may identify a target tissue as tissue corresponding to an artery in the hand. In some implementations, the processor may then determine the diameter of the artery at a plurality of times from the received and/or generated signal, wherein the diameter of the artery changes over time and corresponds to the user's blood pressure. Based on the determination of changes in arterial diameter, in some instances, the processor may then generate a second signal corresponding to the time varying arterial blood pressure of the user based upon changes in diameter of the artery. In some implementations, the processor may determine the arterial pressure from the received signals and/or the arterial diameters using the methods described above with reference to FIG. 1.

In some instances, the function processor may split the sampled data into a plurality of bins, wherein each bin corresponds to a target located at a unique depth in the tissue and represents a magnitude of said reflected signal on said target. In some implementations, the mixer comprises a single Schottky diode, which may also couple the signal generated by the oscillator to the antenna.

With reference to FIG. 4, in some embodiments, the arterial sensor may be located in a handheld shell, a handheld mechanical housing that is designed to removably latch or clamp on to the mobile device. In this embodiment, the electromagnetic sensor may be housed in handheld mechanical housing, 409, separate from the mobile device, which can also act as a mobile device shell to protect the mobile device from concussions. As show in FIG. 4, for example, outer side edges of a mobile device 102 and inner facing surfaces of handheld mechanical housing, 409, contact one another. At or near the points of contact may including clamping or latching structures, which allow the two elements to be held together, and removed from one another. Such mechanical latching structures for mobile devices are well known. In some instances, one or both of the antennas 407, 408 may be connected to the RADAR electronics and wireless transmitter 410. In some embodiments, the housing shell may comprise a communication device to transmit the data to the mobile device and/or some other external device. In some implementations, the housing shell may comprise a power source (e.g., battery) to power the various components of the sensor. In some instances, this communication module may use a technology selected from the group comprising Bluetooth Low Energy (BLE) protocol, Advanced Network Technologies (ANT+) protocol, Near Field Communication (NFC), and any similar data communication.

Mechanical housing 409 is configured to either removably clamp to or contain mobile device 102, and to be held in the hand of a person. Mechanical housing 409 preferably comprises a hard plastic formed layer, to which are attached the antenna 407, 408 and RADAR electronics and wireless transmitter 410. Preferably, the antennas are disposed on an interior surface of the hard plastic that is designed to be opposite the side of the mechanical housing opposing the mobile device and adjacent the side of the mechanical housing opposing the palm of a hand holding the mechanical housing 409. Preferably, the antennae are have their major surfaces aligned in a plane with one another, or in contact with a flat or slightly curved interior surface of the mechanical housing 409. Mechanical housing 409 may have a surface curved to conform to the palm of a user holding the mechanical housing; which positions the antennas slightly closer to arteries in the hand. Processing of the received signal may occur either in the shell 409 or the mobile device 102. One or the other or both comprise a suitable digital data processor, memory, and processing algorithm configured in either hardware or software. The housing 409 has attached thereto a wireless transmitter for transmitting signals to mobile device 102 when housing 409 is removably clamped or latched to or contains mobile device 102. Preferably, this wireless transmitter is configured for transmitting using a Bluetooth specification, generically meaning transmission in the 2400 and 2483.5 MHZ frequency range and transmitting using frequency hopping spread spectrum. However, this wireless transmitter may instead use ANT+; NFC; or some other protocol for very short range low power wireless communications. Typically, Bluetooth signal from the mechanical housing 409 is only strong enough to be received by the mobile device when the mobile device is within about 10 feet of the mechanical housing 409.

The RADAR electronics must be very lower power, with power from 1 uW and 100 mW peak, and average power from 1 uW to 10 mW. Therefore the RADAR electronics (particularly the antenna or antennas) must also be very close to the target artery, not further than 10 centimeters. The RADAR electronics preferably functions in the frequency between 3.1 to 10.6 GHZ, and preferably in a subband of bandwidth of at least 500 MHZ, and more preferably in a subband of bandwidth more than 2 GHz.

In addition to or as an alternative to the RADAR electronics, the mechanical housing 409 may include some other form of electromagnetic receiver for receiving signals, either intended for biometric signals from the body of the person holding the mechanical housing such as the RADAR reflections discussed herein, or environmental signals from the environment in the vicinity of the mechanical housing indicative of the environment around the mechanical hosing 409. The environment in the vicinity may be at least one region of space or surface within 1 kilometer, 0.1 kilometer, 10 meters, 1 meter, or 10 centimeters of the handheld mechanical housing. Obviously, for longer range operations than the biometric sensing noted above, comparably higher pulse transmission intensities are required.

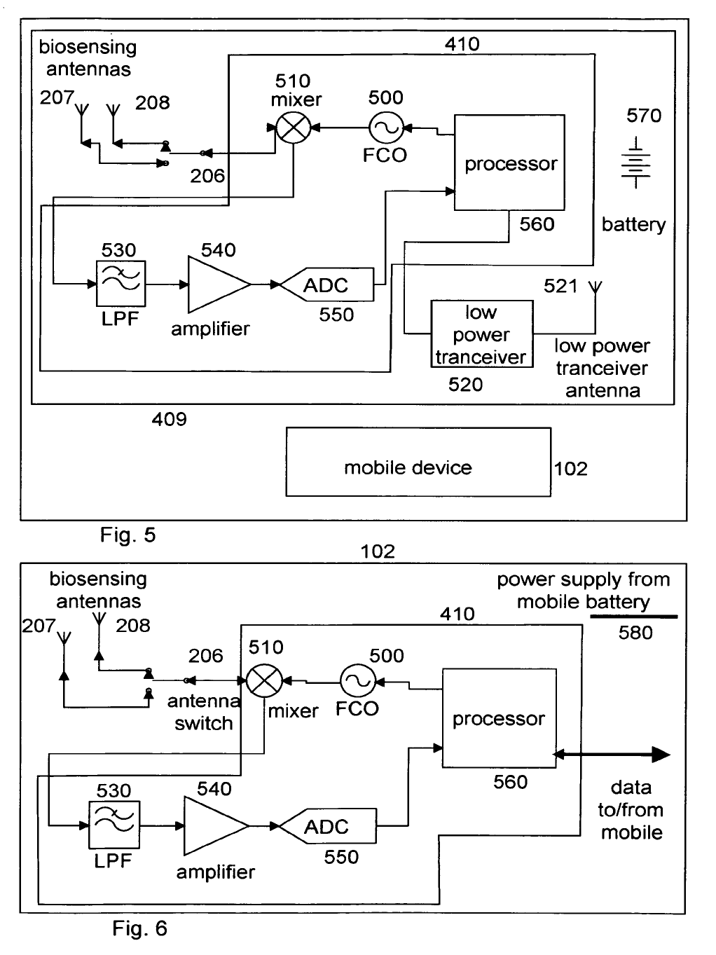

FIG. 5 shows an electrical block diagram of an embodiment of mechanical housing 409 including battery B 570, biometric sensing antennas 207 and 208, antenna selection switch 206, and RADAR circuit 410 comprising frequency controlled oscillator, FCO 500, mixer 510, low pass filter (LPF) 530, amplifier 540, analog to digital converter (ADC) 550, processor 560, lower power transceiver 520, and its associated antenna 521. In this embodiment, the antenna 207, 208 positioned closer to the artery is selected by switch 206 which is controlled by processor 560.

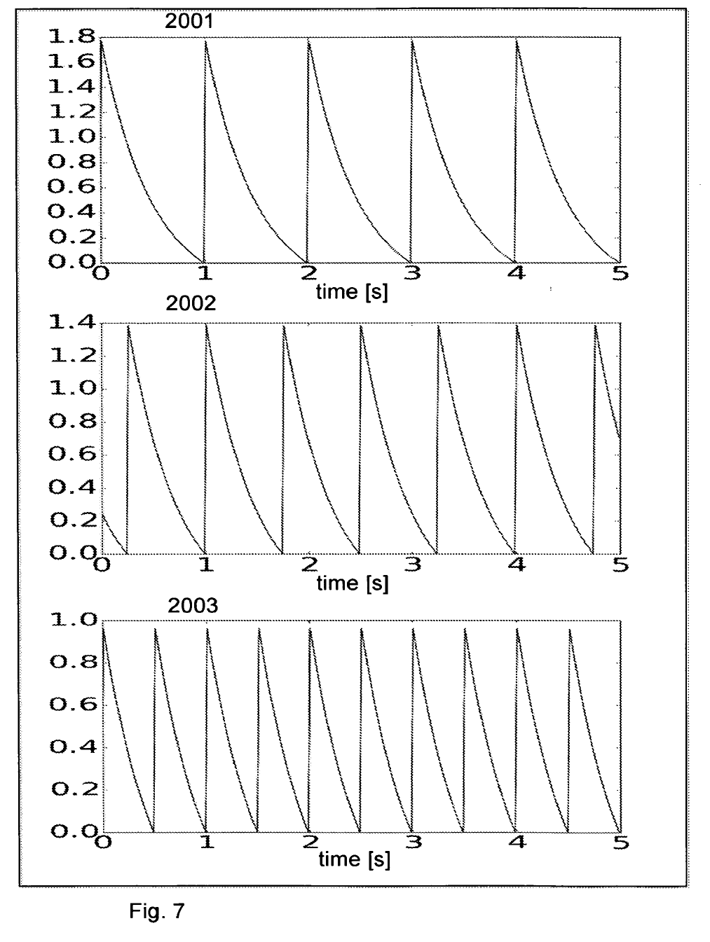

The heart rate, which, for example, can be estimated using a Maximum Likelihood Estimation (MLE) algorithm on the time domain signal resulting from blood pressure variation, can be estimated by the processor 560 and transmitted to the mobile device 102 electronics for further processing and display. Likewise, and estimation of the blood pressure of the user can be transmitted to the mobile device 102 for processing and display. The MLE Algorithm is based on comparing the time domain signal relating the time varying blood pressure with a series of predefined templates. These templates describe the theoretical time dependent blood pressure for the various possible heart rates to be analyzed, for example 171 templates, describing the theoretical blood pressure versus time model for different heart rates from 30 Beats Per Minute (BPM) to 200 BPM, in 1 BPM steps. FIG. 7 shows curves 2001, 2002, 2003, versus time, in seconds. These curves are examples of predetermined templates for heart rates of 60, 80, 120 BPM. The signal containing heart rate information is correlated with a series of the templates. The template resulting in the largest correlation is the maximum likelihood (MLE) template. The heart rate for that template is determined to be the heart rate.

FIG. 6 shows an electrical block diagram of an embodiment of mobile device 102 for use without mechanical housing 409. In this embodiment, mobile device 102 includes biometric sensing antennas 207, 208, antenna switch 206 and RADAR electronics 510 comprising, FCO 500, mixer 510, low pass filter LPF 530, amplifier 540, analog to digital converter ADC 550, and processor 560. Arrows in FIGS. 5 and 6 indicate direction of signal flow. A low pass filter, amplifier, and ADC may reside in FIG. 5 along the path indicated by the dashed line.

Signals resulting from the biometric signals, and environmental signals may be communicated to the mobile device processor via a communication bus, such as SPI, USB, I2C etc. The data processing can be performed in the mobile device, or in the processor 560.

The RADAR 510 and the antennas and switches, may be also used to detect other environmental information. For example, radar can be used to assess the existence and distance of concealed metallic objects, like pipes or electric conduits inside insulating walls, for example, in homes; the ground, natural caves, and artificial and manmade underground regions.

Various inventive systems and methods have been disclosed and are considered part of the invention. Example embodiments of the devices, systems and methods have been described herein. As may be noted elsewhere, these embodiments have been described for illustrative purposes only and are not limiting. Other embodiments are possible and are apparent from the teachings contained herein. The scope of protection sought is defined by the appended claims.

1. A system for determining arterial blood pressure of a user as a function of time, comprising:
a handheld structure comprising at least one of: (1) a handheld device configured to be held in one hand of a person comprising a cellular telephone transceiver and human interface; and (2) a handheld mechanical housing configured to be held in one hand of the person, and designed to removably mechanically attach or clamp onto a handheld device;
a frequency controlled oscillator to generate Radio Frequency (RF) signals;
at least one antenna configured to transmit the RF signals generated by the frequency controlled oscillator and configured to receive RF signals which are reflections of the transmitted RF signals;
a mixer configured to mix at least one of the transmitted RF signals with at least one of the reflected RF signals to generate a modified signal;
an analog-to-digital converter configured to convert the modified signal into digital data thereby providing a digital representation of said modified signal;
at least one processor for processing digital data;
wherein said handheld structure contains said frequency controlled oscillator; said at least one antenna; and said mixer; said analog-to-digital converter, and said at least one processor; and
wherein said system is configured to use said digital representation of said modified signal and a model relating said modified signal to arterial blood pressure, to generate an arterial blood pressure signal indicating arterial blood pressure of the user as a function of time;
said at least one antenna defines a slot;
a feed line in the form of a conductive layer coupling said the RF signals generated by the frequency controlled oscillator to said antenna;
wherein said frequency controlled oscillator is controlled to generate said Radio Frequency (RF) signals in pulses at a repetition rate, and said repetition rate is sufficiently above the heart rate to provide reflection signal data points allowing curve fitting the reflection signal data to form a useful approximation for periodic variations in artery diameter at the periodicity of the heart rate
wherein said frequency controlled oscillator is controlled to generate said Radio Frequency (RF) signals in a frequency range between 3.1 and 10.6 GHz and with a subband of bandwidth of at least 500 MHz;
wherein said RF signals generated by the frequency controlled oscillator have peak power between 1 uW and 100 mW peak, and average power between 1 uW (microWatt) and 10 mW (milliWatt); and
a battery, wherein said battery provides power to said frequency controlled oscillator.

2. The system of claim 1 wherein said handheld device contains said frequency controlled oscillator, said at least one antenna; and said mixer; said analog-to-digital converter; and said at least one processor.

3. The system of claim 1, wherein said oscillator is configured to generate signals having a bandwidth of at least 3 GHz in a frequency range between 3.0 GHz to 10.7 GHz.

4. The system of claim 1, wherein said mixer comprises a single mixer; and wherein said mixer is configured to couple said frequency controlled oscillator to said at least one antenna.

5. A system for determining arterial blood pressure of a user as a function of time, comprising:
a handheld structure comprising at least one of: (1) a handheld device configured to be held in one hand of a person comprising a cellular telephone transceiver and human interface; and (2) a handheld mechanical housing configured to be held in one hand of the person, and designed to removably mechanically attach or clamp onto a handheld device;
a frequency controlled oscillator to generate Radio Frequency (RF) signals;
at least one antenna configured to transmit the RF signals generated by the frequency controlled oscillator and configured to receive RF signals which are reflections of the transmitted RF signals;
a mixer configured to mix at least one of the transmitted RF signals with at least one of the reflected RF signals to generate a modified signal;
an analog-to-digital converter configured to convert the modified signal into digital data thereby providing a digital representation of said modified signal;
at least one processor for processing digital data;
wherein said handheld structure contains said frequency controlled oscillator, said at least one antenna; and said mixer; said analog-to-digital converter, and said at least one processor; and
wherein said system is configured to use said digital representation of said modified signal and a model relating said modified signal to arterial blood pressure, to generate an arterial blood pressure signal indicating arterial blood pressure of the user as a function of time; and
wherein said at least one antenna comprises a first antenna and a second antenna, said first antenna is located in said handheld structure relative to an artery in a left hand of the user so that it senses arterial diameter when said system is held in the left hand of said user, and said second antenna is located in said handheld structure relative to an artery in a right hand of the user so that it senses arterial diameter when said system is held in the right hand of said user.

6. The system of claim 1 wherein said system is configured to transmit said arterial blood pressure signal indicating arterial blood pressure of the user as a function of time over a cellular network.

7. The system of claim 1 further comprising a heart rate determination algorithm designed to determine heart rate of a user, wherein said heart rate determination algorithm is designed to determine heart rate of a user from said digital representation of said modified signal and at least one model function for heart rate, wherein said algorithm is implemented in at least one of software and hardware.

8. The system of claim 1 comprising said handheld mechanical housing and wherein said handheld mechanical housing contains said frequency controlled oscillator; said at least one antenna; and said mixer, and a transceiver designed to wirelessly communicate with said handheld device.

9. The system of claim 8 comprising both said handheld mechanical housing and said handheld device; and
wherein said handheld mechanical housing contains said frequency controlled oscillator, said at least one antenna, said mixer, and a low power transceiver designed to wirelessly communicate with said handheld device.

10. The system of claim 9 wherein said low power transceiver is designed to transmit in at least one of the following frequency bands: 6.765 to 6.795 MHZ; 13.553 to 13.567 MHZ; 26.957 to 27.283 MHZ; 40.660 to 40.700 MHZ; 433.050 to 434.790 MHZ; 902.000 to 928.000 MHZ; 2.400 to 2.500 GHz; 5.725 to 5.875 GHz; 24.000 to 24.250 GHz; 61.000 to 61.500 GHz; 122.000 to 123.000 GHz; and 244.000 to 246.000 GHz.

11. The system of claim 9 wherein said low power transceiver is configured to transmit either said modified signal or said digital representation of said modified signal to said handheld device, when said handheld device is removably mechanically clamped or latched onto or contained in said handheld mechanical housing.

12. The system of claim 1 wherein said system is designed to estimate distance of the antenna or antennas from the skin using the amplitude and phase of the signal reflected from skin, and to compensate determination of arterial blood pressure based upon there upon.

13. The system of claim 5 wherein said handheld structure has a relatively long dimension, a first end along said relatively long dimension, a second end along said relatively long dimension, said first antennae is disposed closer to said first end than said second end, and said second antennae is disposed relatively closer to said second end than said first end.

14. The system of claim 1 further comprising an elliptical disk at a termination of said feed line adjacent said slot.

15. The system of claim 1 further comprising:
a bottom dielectric sheet and a top dielectric sheet stacked upon one another;
wherein metal defining said slot resides on a top surface of said top dielectric;
wherein said feed line resides beneath said top dielectric sheet; and
further comprising a detector diode located on said top dielectric sheet.

16. A method for determining arterial blood pressure of a user as a function of time, comprising:
powering with a battery, a handheld structure comprising at least one of: (1) a handheld device configured to be held in one hand of a person comprising a cellular telephone transceiver and human interface; and (2) a handheld mechanical housing configured to be held in one hand of the person, and designed to removably mechanically clamp or latch onto a handheld device; a frequency controlled oscillator to generate RF signals; at least one antenna configured to transmit the RF signals generated by the frequency controlled oscillator and configured to receive RF signals which are reflections of the transmitted RF signals; a mixer configured to mix at least one of the transmitted RF signals with at least one of the reflected RF signals to generate a modified signal; an analog-to-digital converter configured to convert the modified signal into digital data thereby providing a digital representation of said modified signal; at least one processor for processing digital data; wherein said handheld structure contains said frequency controlled oscillator; said at least one antenna; and said mixer, said analog-to-digital converter; and said at least one processor; and
using said digital representation of said modified signal and a model relating said modified signal to arterial blood pressure, to generate an arterial blood pressure signal indicating arterial blood pressure of the user as a function of time;
wherein said at least one antenna defines a slot;
coupling said the RF signals generated by the frequency controlled oscillator to said antenna via a feed line in the form of a conductive layer,
controlling said frequency controlled oscillator to generate said RF signals in pulses at a repetition rate, and said repetition rate is sufficiently above the heart rate to provide reflection signal data points allowing curve fitting the reflection signal data to form a useful approximation for periodic variations in artery diameter at the periodicity of the heart rate
controlling said frequency controlled oscillator to generate said RF signals in a frequency range between 3.1 and 10.6 GHz and with a subband of bandwidth of at least 500 MHz;
wherein said RF signals generated by the frequency controlled oscillator have peak power between 1 uW and 100 mW peak, and average power between 1 uW (microWatt) and 10 mW (milliWatt); and
wherein said battery provides power to said frequency controlled oscillator.

17. The method of claim 16 further comprising determining heart rate of a user, using a heart rate determination algorithm designed to determine heart rate of a user, wherein said heart rate determination algorithm is designed to determine heart rate of a user from said digital representation of said modified signal and at least one model function for heart rate, wherein said algorithm is implemented in at least one of software and hardware.

18. A system for determining arterial blood pressure of a user as a function of time, comprising:
a handheld structure comprising at least one of: (1) a handheld device configured to be held in one hand of a person comprising a cellular telephone transceiver and human interface; and (2) a handheld mechanical housing configured to be held in one hand of the person, and designed to removably mechanically attach or clamp onto a handheld device;
a frequency controlled oscillator to generate Radio Frequency (RF) signals;
at least one antenna configured to transmit the RF signals generated by the frequency controlled oscillator and configured to receive RF signals which are reflections of the transmitted RF signals;
a mixer configured to mix at least one of the transmitted RF signals with at least one of the reflected RF signals to generate a modified signal;
an analog-to-digital converter configured to convert the modified signal into digital data thereby providing a digital representation of said modified signal;
at least one processor for processing digital data; and
wherein said at least one antenna comprises a first antenna and a second antenna, said first antenna is located in said handheld structure relative to an artery in a left hand of the user so that it senses arterial diameter when said system is held in the left hand of said user, and said second antenna is located in said handheld structure relative to an artery in a right hand of the user so that it senses arterial diameter when said system is held in the right hand of said user.

19. The system of claim 18 wherein metal forming said first antenna and said second antenna are disposed on the same surface of a dielectric substrate.

20. The system of claim 18 further comprising an antenna selection switch coupled to said mixer, said first antenna, and said second antenna, wherein said antenna selection switch is controlled by said at least one processor.
